(19) United States
(12) Patent Application Publication
(10) Pub. No.: US 2021/0343414 A1
(43) Pub. Date: Nov. 4, 2021

(54) METHODS AND APPARATUS FOR PHENOTYPE-DRIVEN CLINICAL GENOMICS USING A LIKELIHOOD RATIO PARADIGM

(71) Applicant: The Jackson Laboratory, Bar Harbor, ME (US)

(72) Inventor: Peter N. Robinson, Robinson, ME (US)

(73) Assignee: The Jackson Laboratory, Bar Harbor, ME (US)

(21) Appl. No.: 17/285,435

(22) PCT Filed: Oct. 21, 2019
(86) PCT No.: PCT/US2019/057155

§ 371 Date: Apr. 14, 2021

Related U.S. Application Data
(60) Provisional application No. 62/748,898, filed on Oct. 22, 2018.

(51) Int. Cl. G16H 50/20; G16H 50/30; G16H 50/50

(52) U.S. Cl. CPC G16H 50/20; G16H 50/50; G16H 50/30

(57) ABSTRACT

Methods and apparatus for providing clinical decision support. The method comprises receiving phenotype information for a patient, determining a likelihood ratio for each of the phenotype features included in the received phenotype information with respect to each of a plurality of diseases, determining, based on the likelihood ratio for each of the phenotype features, a composite likelihood ratio for each of the plurality of diseases, ranking the plurality of diseases based, at least in part, on the determined composite likelihood ratios, and displaying at least some of the ranked plurality of diseases.

### BACKGROUND

Phenotype-driven prioritization of candidate genes and diseases is a well-established approach towards genomic diagnostics in rare disease. Some conventional approaches use the Human Phenotype Ontology (HPO) for annotating the set of phenotypic abnormalities observed in the individual being investigated by exome or genome sequencing. A recent version of the HPO contains 13,726 terms arranged as a directed acyclic graph in which edges represent subclass relations; 13,559 of these terms represent phenotypic abnormalities. For instance, Abnormal renal cortex morphology is a subclass of Abnormal renal morphology. The HPO project additionally provides computational disease models of 7074 rare diseases that are constructed from HPO terms and metadata that define the diseases based on the phenotypic abnormalities that characterize them, their modes of inheritance, and in many cases the age of onset of diseases or phenotypic features and the overall frequencies of features in a disease. For instance, type 7 Meckel syndrome is characterized by Patent ductus arteriosus (HP:0001643) with a frequency of two of seven patients with antenatal onset.

### SUMMARY

The present disclosure provides, in some aspects, a clinical decision support tool that evaluates the probability that a patient has a particular disease based on a likelihood ratio analysis of observed patient phenotypes and/or genotypes. In particular, some embodiments are directed to an approach towards genomic diagnostics that exploits the clinical likelihood ratio framework to provide an estimate of the posttest probability of candidate diagnoses as well as the odds ratio for each observed phenotype and the predicted pathogenicity of observed genetic variants, thereby providing clinicians with a result that is interpretable with respect to the contribution of each individual phenotypic abnormality. The odds ratio for the genetic variant additionally provides a measure of the tendency of the gene to harbor rare, predicted pathogenic variants in the general population.

Some embodiments are directed to a clinical decision support system comprising at least one computer processor and at least one storage device having stored thereon, a plurality of computer-readable instructions that, when executed by the at least one computer processor, performs a method. The method comprises receiving phenotype information for a patient, determining a likelihood ratio for each of the phenotype features included in the received phenotype information with respect to each of a plurality of diseases, determining, based on the likelihood ratio for each of the phenotype features, a composite likelihood ratio for each of the plurality of diseases, ranking the plurality of diseases based, at least in part, on the determined composite likelihood ratios, and displaying at least some of the ranked plurality of diseases.

Some embodiments are directed to a method of providing clinical decision support. The method comprises receiving phenotype information for a patient, determining a likelihood ratio for each of the phenotype features included in the received phenotype information with respect to each of a plurality of diseases, determining, based on the likelihood ratio for each of the phenotype features, a composite likelihood ratio for each of the plurality of diseases, ranking the plurality of diseases based, at least in part, on the determined composite likelihood ratios, and displaying at least some of the ranked plurality of diseases.

Some embodiments are directed to a non-transitory computer readable medium encoded with a plurality of instructions that, when executed by at least one computer processor perform a method. The method comprises receiving phenotype information for a patient, determining a likelihood ratio for each of the phenotype features included in the received phenotype information with respect to each of a plurality of diseases, determining, based on the likelihood ratio for each of the phenotype features, a composite likelihood ratio for each of the plurality of diseases, ranking the plurality of diseases based, at least in part, on the determined composite likelihood ratios, and displaying at least some of the ranked plurality of diseases.

It should be appreciated that all combinations of the foregoing concepts and additional concepts discussed in greater detail below (provided such concepts are not mutually inconsistent) are contemplated as being part of the inventive subject matter disclosed herein.

### BRIEF DESCRIPTION OF THE DRAWINGS

Various non-limiting embodiments of the technology will be described with reference to the following figures. It should be appreciated that the figures are not necessarily drawn to scale.

FIG. 1 illustrates a process for providing clinical decision support in accordance with some embodiments;

FIG. 2 illustrates a process for computing a posttest probability that a patient has a particular disease in accordance with some embodiments;

FIGS. 3A-3C illustrate information for the top three ranked disease candidates given an input set of phenotypic features for a patient using the techniques described herein in accordance with some embodiments;

FIGS. 4A-C illustrate information for the top three ranked disease candidates given a different input set of phenotypic features for a patient using the techniques described herein in accordance with some embodiments;

FIG. 5 illustrates information for a top ranked disease candidate given an input set of phenotypic features for a patient using the techniques described herein in accordance with some embodiments;

FIG. 6 illustrates results of a simulation using different numbers of phenotype terms in accordance with some embodiments; and

FIG. 7 schematically illustrates components of a computer-based system on which some embodiments may be implemented.

### DETAILED DESCRIPTION

Exome sequencing and genome sequencing are techniques for rapid sequencing of large amounts of DNA, and may be used to test for genetic disorders. In exome sequencing, all of the portions of DNA in a person's genome that provide instructions for making proteins (called exons) are sequenced. Exome sequencing allows variants in the protein-coding region of any gene to be identified. In genome sequencing, the order of all nucleotides in an individual's DNA is determined and variants in any part of the genome may be identified.

Exome and genome sequencing typically reveal tens or hundreds of variants that are predicted to be deleterious by common computational frameworks, and therefore the analysis of such data generally applies some additional criterion to prioritize genes. Phenotypic approaches compare the observed phenotypic abnormalities of the person being investigated with computational gene models and search for genes that both harbor a predicted pathogenic variant and also are associated with diseases whose phenotypic abnormalities (e.g., clinical signs, symptoms, or other abnormalities observed as part of a medical examination) are compatible with those observed for a patient. The inventors have recognized that current techniques for phenotype-driven genomic diagnostics have a number of shortcomings that represent impediments to the successful implementation of genomic testing outside of specialist centers. For example, conventional approaches typically present results as an ordered list of candidate genes or diseases; yet if the overall success rate of genomic diagnostics of around 50% or less is considered, one may expect that in many cases, the gene at rank one is actually not a good candidate. To this end, some embodiments are directed to a computational technique for providing a measure of how good the top predictions are. Additionally, the inventors have recognized that approaches that provide clinical users with information to understand the reasons for the computational predictions would make for a more useful clinical decision support tool for such users.

Some embodiments of the technology described herein relate to a computational technique that applies a clinical likelihood ratio (LR) framework to phenotype-driven genomic diagnostics to address at least some of the shortcomings of prior techniques. A likelihood ratio is defined as the probability of a given test result in an individual with the target disorder divided by the probability of that same result in an individual without the target disorder. The LR framework described herein allows multiple test results to be combined by multiplying the individual ratios, and also relates the pretest probability to the posttest probability in a way that can be used to guide clinical decision making. The clinical LR framework as described herein enables a phenotype- and/or genotype-based computational decision support system to assess the relative merits of specific diseases in a differential diagnosis that can encompass hundreds or thousands of diseases.

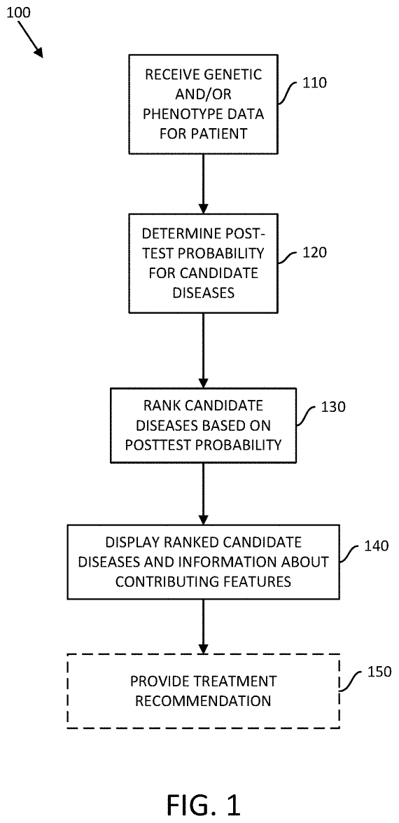

FIG. 1 illustrates a process 100 for providing clinical decision support in accordance with some embodiments. In act 110, genetic data and/or phenotype data for a patient are received. For example, a user interface may be presented to a user and the user may enter at least some of the genetic data and/or phenotype data into the user interface. At least some of the genetic data and/or phenotype data may be provided in some other way for processing. For example, a sample collected from the patient may be assayed and genetic data for the patient may be determined based on the assay. The determined genetic data may be provided as input to one or more of the analysis techniques, described more detail below. In some embodiments, the received phenotype data may include one or more HPO features or terms that describe a particular phenotype in the computational disease models of the HPO project.

Process 100 then proceeds to act 120, where the received phenotype and/or genotype information is used to determine a posttest probability for each of a plurality of candidate diseases. The posttest probability is a measure of how likely it is that the patient has the disease given the input set of genotype and/or phenotype features. Embodiments of the technology described herein use a likelihood ratio analysis paradigm to determine the posttest probabilities. Examples of how the likelihood ratios are computed in accordance with some embodiments are described in more detail below. Process 100 then proceeds to act 130, where the plurality of candidate diseases are ranked based on the determined posttest probabilities. For example, candidate diseases with a higher posttest probability may be ranked higher (the patient is more likely to have the disease) than candidate diseases with lower posttest probabilities.

Process 100 then proceeds to act 140, where at least some of the ranked candidate diseases and information indicating a degree to which particular genotype and/or phenotype features contributed to the overall posttest probability are displayed to a user. Although some conventional phenotype-based clinical genomics techniques may provide a list of possible candidate diseases, the probabilities of the patient having each of the candidate diseases and information describing which features or factors contributed more or less strongly to the overall probability are not typically calculated or shown to the user. The inventors have recognized that providing information on a user interface that enables clinicians to understand why a candidate disease is ranked high and providing information about what features contributed to the high ranking, results in a more effective clinical decision support tool for the clinician. For example, by identifying particular phenotypic features that significantly positively or negatively affect the posttest probability, the clinician may verify that the user has those phenotypic characteristics to ensure that the disease diagnosis is accurate. Process 100 then optionally proceeds to act 150, where a recommendation for clinical management (e.g., a treatment recommendation) determined based, at least in part, on the ranked list of candidate diseases may be provided, for example, on a user interface.

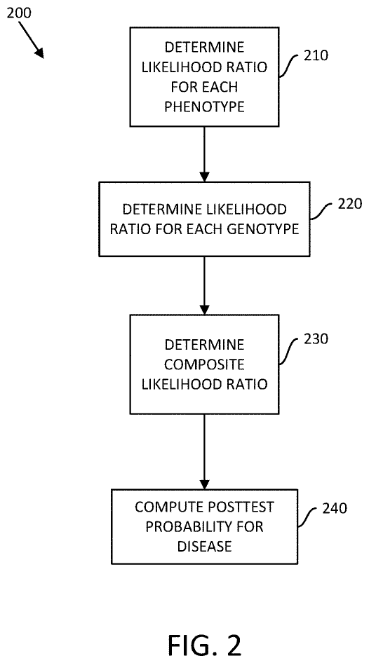

FIG. 2 illustrates a process 200 for determining a posttest probability for a disease given an input set of genotype and/or phenotype features in accordance with some embodiments. In act 210, a likelihood ratio is determined for each of the phenotype features provided as input to the process. Example techniques for calculating a likelihood ratio for a feature hᵢ is described in more detail below. Process 200 then proceeds to act 220, where, if genetic information is provided as input, a likelihood ratio is determined for each genotype included in the genetic information. For example, particular diseases may have known associations with particular gene variants. As used herein, the “genotype” refers to the overall count of variants observed at a given gene. For some diseases (e.g., with autosomal dominant inheritance), a single (heterozygous) variant in a gene can trigger disease. For other diseases (e.g., with autosomal recessive inheritance), two variants are required, either with a homozygous genotype (two copies of the same variant on the maternal and paternal chromosome) or two distinct variants in the same gene (compound heterozygous genotype). Accordingly, if the patient has a particular genetic variant and genotype associated with a particular disease, that may be indicative of the patient having the disease. Alternatively, if the patient does not have the particular genetic variant, that may be indicative of the patient not having the particular disease. Process 200 then proceeds to act 230, where a composite likelihood ratio is determined. In embodiments in which only phenotypic information is provided as input, the composite likelihood ratio may be based on the likelihood ratios determined for the individual phenotype features provided as input. In embodiments that include both phenotypic and genetic information as input, the composite likelihood ratio may be further based, at least in part, on the likelihood ratio(s) determined for each genotype. Process 200 then proceeds to act 240, where the posttest probability for a disease is determined based on the composite likelihood ratio.

### Likelihood Ratio-Based Model

A LR-based model of the clinical examination of a patient being investigated for a suspected but unknown Mendelian disorder may be defined as follows. Each recorded phenotypic observation is defined as a clinical test. The set of genetic data determined, for example, from an exome, genome, or gene panel experiment in addition to a list of ontology terms (e.g., HPO terms) that describe the phenotypic abnormalities of the person being investigated (in the following, the person being investigated is referred to as a “proband”) are used as input to the likelihood ratio analysis. An “odds ratio” having a numerator and a denominator in the LR-based model may be used to express the odds that a disease will be present given that a phenotype is observed compared to the odds that the phenotype is not observed. For the numerator, the probability of a person with disease D having a phenotypic abnormality encoded by HPO term hᵢ, denoted as f_{i,D}, is recorded in the computational disease models of the HPO project (or some other suitable database) based on literature biocuration, or may be taken to be 100% if more detailed information is not available. For many diseases and features, an overall frequency of the feature is known; for instance, 19/437 (˜4%) of persons with neurofibromatosis type 1 have seizures. On the other hand, 338/442 (˜87%) of individuals with this disease have multiple cafe-au-lait spots.

The denominator of the odds ratio is the probability of the phenotypic feature if the proband does not have the disease in question. Although it may be difficult to calculate this quantity for each of the approximately 13,000 phenotypic abnormalities of the HPO in the general population, a tractable and not unrealistic model may be that any proband being investigated by genomic diagnostics has some genetic disease. Taking this assumption, the denominator of the likelihood ratio may be calculated using the overall prevalence of HPO feature hᵢ in genetic diseases other than D. For instance, if disease D and thirteen other diseases of the total of 7000 diseases in the HPO database are characterized by feature hᵢ and an equal pretest probability is assumed for all diseases, then the probability of the proband having feature hᵢ if the proband is not affected by disease D is 13/7000.

### Likelihood Ratio

The likelihood ratio (LR) is a measure used in accordance with some embodiments to compute the accuracy of tests. LR is defined as the probability of a given test result in a patient with the target disorder divided by the probability of that same result in a person without the target disorder. The LR of a positive test result (LW⁺) is defined as the probability that an individual with the target disorder Dⱼ has a positive test result x divided by probability that an individual without the target disorder (Dⱼ) has a positive test result:

$${LR}^{+} = \frac{sensitivity}{1 - specificity} = \frac{p ( x \vert {D}_{j} )}{p ( x \vert ⫬ {D}_{j} )} \qquad ( 1 )$$

where the sensitivity (true positive rate) of the test is the proportion of individuals with disease Dⱼ who are correctly identified and the specificity or true negative rate is the proportion of individuals without disease Dⱼ who are correctly identified as unaffected. The definition of the likelihood ratio can be extended to multiple tests. Suppose X=(x₁, x₂, . . . , xₙ) is an array of n test results. Under the assumption that the tests are independent, the LR is

$$LR ( X ) = \frac{P ( X \vert {D}_{j} )}{P ( X \vert ⫬ {D}_{j} )} = \frac{P ( {x}_{1} , {x}_{2} , … , {x}_{n} \vert {D}_{j} )}{P ( {x}_{1} , {x}_{2} , … , {x}_{n} \vert ⫬ {D}_{j} )} = {\prod }_{i = 1}^{n} \frac{P ( {x}_{i} \vert {D}_{j} )}{P ( {x}_{i} \vert ⫬ {D}_{j} )} \qquad ( 2 )$$

The likelihood ratio of a negative test result LR⁻=(1−sensitivity)/specificity. The following considerations may be performed analogously if negative test results are used (e.g., the phenotypic abnormality in question was ruled out in the proband).

The posttest probability refers to the probability that a patient has a disease given the information from test results X and can then be calculated as

$$P ( {D}_{j} \vert X ) = \frac{pLR ( X )}{( 1 - p ) + pLR ( X )} \qquad ( 3 )$$

where p is the pretest probability of Dⱼ. Depending on the cohort, the pretest probability can be defined as the population prevalence of the disease or may be defined by some other estimate of the frequency of the disease in the cohort being tested.

### Likelihood Ratio for Phenotypes

The signs and symptoms and other phenotypic abnormalities of probands being investigated using some embodiments are represented, for example, using terms of the Human Phenotype Ontology (HPO), which provides a structured, comprehensive and well-defined set of classes (terms) describing human phenotypic abnormalities. The clinical encounter that results in a set of n phenotypic observations is modeled and encoded as HPO terms h₁, h₂, . . . , hₙ. The likelihood ratio of each phenotype term with respect to a specific disease Dⱼ is defined as:

$$LR ( {h}_{i} ) = \frac{P ( {h}_{i} \vert {D}_{j} )}{P ( {h}_{i} \vert ⫬ {D}_{j} )} \qquad ( 4 )$$

assuming that the tests are independent and the likelihood ratio of the n HPO terms are obtained from equation (2).

The Probability of Having Phenotypic Abnormality hᵢ Given a Disease Dⱼ

In some embodiments, the numerator of equation (4) is determined based on the relationship of term hᵢ to the set of phenotype terms with which disease Dⱼ is annotated. Four cases (i)-(iv), described in more detail below are evaluated in some embodiments to determine the numerator of equation (4).

(i) hᵢ is identical to one of the terms to which Dⱼ is annotated in the database.

In this case, P(hᵢ|Dⱼ)=f_{i,Dj}, that is, the frequency of the phenotypic feature hᵢ amongst individuals with disease Dⱼ. For instance, if the disease model for Dⱼ is based on a study in which 7 of 10 persons with Dⱼ had feature hᵢ, then f_{i,Dj}=0.7. If no information is available about the frequency of hᵢ, some embodiments may define f_{i,Dj}=1 (or some other default value representing the average frequency of features in a disease).

(ii) hᵢ is an ancestor of one or more of the terms to which Dⱼ is annotated in the database. Because of the annotation propagation rule of subclass hierarchies in ontologies, Dⱼ is implicitly annotated to all of the ancestors of the set of annotating terms. For instance, if the computational disease model of some disease D includes the HPO term Polar cataract (HP:0010696) then the disease is implicitly annotated to the parent term Cataract (HP:0000518). For example, any person with a polar cataract necessarily also more generally may be considered to have a cataract. By extension, this relation is also true of more distant descendants of the term. Accordingly, in some embodiments the probability of a term hᵢ that is annotated to an ancestor of any term that explicitly annotates disease Dⱼ is defined as:

$${h}_{i} = {max}_{{h}_{i} ∈ anc ( {h}_{j} ) ⋀ {h}_{j} ∈ annot ( {D}_{j} )} {f}_{j , {D}_{j}} \qquad ( 5 )$$

where anc(hⱼ) is a function that returns the set of all ancestors of term hⱼ and annot(Dⱼ) is a function that returns the set of all HPO terms that explicitly annotate disease Dⱼ.

(iii) hᵢ is a descendant of one or more of the terms to which Dⱼ is annotated.

In this case, hᵢ is a descendant (e.g., a specific subclass of) term hⱼ of disease Dⱼ. For instance, disease Dⱼ might be annotated to Syncope (HP:0001279), and the query term hᵢ may be Orthostatic syncope (HP:0012670), which is a child term of Syncope in the ontology. In addition, Syncope has two other child terms, Carotid sinus syncope (HP:0012669) and Vasovagal syncope (HP:0012668). In accordance with some embodiments, the frequency of Syncope in disease Dⱼ (e.g., 0.72) may be weighted using a weighting factor of one divided by the total number of child terms of hⱼ (so in the example above a frequency of 0.72×⅓=0.24 would be used). If hᵢ is not a direct child of hⱼ, then the definition may be applied recursively. For instance, if term hⱼ has three children terms including hₖ and hᵢ is identical with one of the two child terms of hₖ, then the frequency may be weighted by ⅓×½=⅙).

(iv) hᵢ is neither an ancestor or descendant of any term to which Dⱼ is annotated in the database.

In this case, hᵢ is unrelated to any of the terms that characterize disease Dⱼ. For instance, if disease Dⱼ is characterized only by cardiovascular abnormalities, then the finding of hearing difficulties (HPO term hᵢ) may be considered to be unrelated to disease Dⱼ. In this case, term hᵢ is connected only by the root phenotype term to any of the terms of Dⱼ, and one would have to ascend all the way to the root of the phenotype ontology to find the common ancestor of Hearing impairment (HP:0000365) and a cardiovascular anomaly such as Ventricular septal defect (HP:0001629). In principle, such findings could be modeled using the population prevalence because, for example, a finding such as myopia is relatively common in the general population and can also be found in persons with Mendelian disease without necessarily being causally related to the disease. However, in practice, reliable data concerning the population prevalence of the phenotypic findings represented by the approximately 13,000 HPO terms may not available. Accordingly, in some embodiments, this probability may be set to an arbitrary small number (e.g., 1:20,000 for the analysis described in more detail below).

### The Probability of Having Phenotypic Abnormality hi if Disease Dj is not Present

The denominator of equation (4) specifies the probability of the test result given that the proband does not have some disease Dⱼ. The probability may be difficult to calculate for the general population for reasons similar to those described above. However, some embodiments are configured to estimate this probability if it is assumed that all persons being tested have some (unknown) Mendelian disorder by simply summing over the overall frequency of a feature in the entire HPO corpus (with N diseases).

$$P ( {h}_{i} \vert ⫬ {D}_{j} ) = \frac{1}{N - 1} {\sum }_{k \neq j} P ( {h}_{i} \vert {D}_{k} ) = \frac{1}{N - 1} {\sum }_{k \neq j} {f}_{i , {D}_{k}} \qquad ( 6 )$$

Equation (6) may be calculated separately for each of the N diseases. Alternatively, because in practice, equation (6) may be summed over a relatively large number of diseases (e.g., >7000 diseases), some embodiments use the following approximation that allows for precalculating P (hᵢ|¬Dⱼ) for an arbitrary disease Dⱼ.

$$P ( {h}_{i} \vert ⫬ {D}_{j} ) = \frac{1}{( N - 1 )} {\sum }_{k \neq j} {f}_{i , {D}_{k}} \approx \frac{1}{N} {\sum }_{k = 1}^{N} {f}_{i , {D}_{k}} \qquad ( 7 )$$

### Likelihood Ratio for Genotypes

Some embodiments that predict the relevance of any given genotype make use of the following concepts. There is a true but unobservable pathogenicity, defined as a deleterious effect of a genetic variant on the biochemical function of a gene and the gene product it encodes that leads to disease. The pathogenicity prediction of a variant is made on the basis of a computational pathogenicity score that ranges from 0 (predicted benign) to 1 (maximum pathogenicity prediction). The model described herein posits two distributions that enable for calculating the likelihoods of an observed genotype given that the sequenced individual has the disease (D) as compared to the situation in which the individual does not have the disease in question and the variants originate from population background (B). A score for any variant in the coding exome or at the highly conserved dinucleotide sequences at either end of introns is used in some embodiments. The estimated population frequencies of variants are derived from, for example, the gnomAD database or other databases that contain information on the population frequencies of genetic variants.

Some embodiments depend on the assumed mode of inheritance of the disease. For autosomal dominant (AD) diseases, the ratio of an observed genotype (G) given that it is disease-causing (i.e., the sequenced individual has disease D) or not (i.e., the sequenced individual does not have disease D) may be of interest. Assuming n observed variants (v₁, v₂, . . . , vₙ) in gene g, with calculated pathogenicity scores s(vᵢ) for i ∈ {1, . . . , n}. For simplicity, it is assumed that the n variants have been arranged such that s(v₁)≥s(v₂)≥ . . . ≥s(vₙ).

It is noted that the majority of variants classified as pathogenic in ClinVar are assigned a pathogenicity score above some arbitrary threshold such as 0.8 (for instance, 98.7% of variants classified as pathogenic in ClinVar are above the threshold of 0.8), with the assumption that the great majority of variants whose score is below the threshold are benign and that the great majority of pathogenic variants will have a score above the threshold (as will additional neutral variants that cannot be distinguished computationally from the pathogenic variants). For the purposes of assessing and scoring candidate variants, some embodiments divide the pathogenicity score distribution into two bins N and P, with bin N representing the predicted non-pathogenic bin and having a range of pathogenicity scores of [0, 0.8], and bin P representing the predicted pathogenic bin with pathogenicity scores of [0.8, 1]. Although in reality there is no strict division in pathogenicity scores between neutral and disease-causing variants, some embodiments use the binning as a way of downweighting variants in genes that often show predicted pathogenic variants and tend to be frequently found as false positives in exome sequencing results, such as many mucin and HLA genes.

Some embodiments model the expected counts of observed alleles in bin P as Poisson distributions, using separate distributions for the case that a variation in a given gene is disease-causing or not. For an autosomal dominant disease, one heterozygous disease causing variant is expected, and so λ^{P,D}=1; for autosomal recessive diseases, λ^{P,D}=2. The probability of observing a variant in bin P in a gene that is not related to the disease may be estimated based on the frequency of such variants in the general population; this probability may be denoted as λ^{P,B}. Different genes have different distributions of predicted pathogenic variants in the general population. The observation of a predicted pathogenic variant in a gene that has a low frequency of such variants in the general population may be interpreted as providing support for the variant being a true-positive. λ^{P,B} may be calculated based on available population frequency data from the gnomAD resource by summing up the frequencies of individual variants under the independence assumption. Although this approach may overestimate the overall frequency of variants per exome/genome, it is used in some embodiments to downweight affected genes as shown below. The function that returns the predicted pathogenicity of a variant is denoted as “path” and the function that returns the maximum population frequency of a variant is denoted as “freq.” This parameter is calculated separately for each gene. The fact that variant i is assigned to gene g is represented as vᵢ ∈ g.

$${λ}^{P , {B}_{g}} = {\sum }_{{path ( {v}_{i} ) ∈ P}_{{v}_{i} \hat{∈} g}} freq ( {v}_{i} ) + ɛ \qquad ( 8 )$$

The parameter λ^{P,B}^{_{g}} is the expected count of variants in gene g whose pathogenicity score is in bin P. A small number (e.g., ε=10⁻⁵) may be added to the sum to avoid division by zero in subsequent steps because some genes may not display any variants in bin P in the population data. For a gene associated with an autosomal dominant disease, the calculation proceeds as follows. Suppose there is a disease Dj which is associated with mutations in gene g, one predicted-pathogenic variant v′in bin P, and k other predicted non-pathogenic variants in bin N (variant v′ thus has a higher pathogenicity score than any of the k other variants). The model according to some embodiments assumes that any variants in bin N are unrelated to the disease and have the same probability whether or not gene g is causally related to the disease. The genotype observed for gene g is symbolized as gt(g).

$$LR ( gt ( g ) ) = \frac{Pr ( gt ( g ) \vert {D}_{j} )}{Pr ( gt ( g ) \vert ⫬ {D}_{j} )} = \frac{Pr ( {v}^{′} \vert {D}_{j} )}{Pr ( {v}^{′} \vert ⫬ {D}_{j} )} \times {Π}_{{i}_{{v}_{i} \neq {v}^{′}}} \frac{Pr ( {v}_{i} ) \vert ⫬ {D}_{j}}{Pr ( {v}_{i} ) \vert ⫬ {D}_{j}} = \frac{Pr ( {v}^{′} \vert {D}_{j} )}{Pr ( {v}^{′} \vert ⫬ {D}_{j} )}$$

The process by which a variant or variants lead to disease by a compound distribution may be modeled. A Poisson distribution models the number of variants observed whose pathogenicity score is in bin P, and a Bernoulli distribution with parameter p=s(v′) determines the probability that the allele is disease causing. Thus, let {Xₙ} be a sequence of mutually independent random variables each of which can take on the value of 0 (for not disease-causing) or 1 (for disease-causing). The sum of N such variables is S_{N}=X₁+X₂+ . . . Xₙ, where S_{N} represents the count of truly pathogenic alleles (e.g., it is expected that S_{N}=1 for autosomal dominant and S_{N}=2 for autosomal recessive diseases).

This leads to the compound distribution

Pr{Sₙ=k}=Binom (k;n, p)Pois (k; λ) (9)

It can be shown that this is equivalent to a Poisson distribution with parameter λp. Therefore, to calculate the likelihood ratio, the parameters λ^{P,D} and Δ^{P,Bg} as well as p=s(vᵢ) may be substituted as follows.

$$LR ( g ) = \frac{Pr ( {v}^{′} \vert D )}{Pr ( {v}^{′} \vert B )} = \frac{Pois ( 1 ; s ( {v}_{i} ) \cdot {λ}^{P , D} )}{Pois ( 1 ; s ( {v}_{i} ) \cdot {λ}^{P , {B}_{g}} )} \qquad ( 10 )$$

This will have the effect of favoring genes with a single variant in bin P that has a maximal pathogenicity score (s(v′)=1) and that has a minimal frequency of bin P variants in the population (if this is the case, then λ^{P,Bg}=ε LR(g)≈36788).

If k>1 variants in a gene g are observed in bin P, then the average pathogenicity score s^{avg} of the variants may be modeled as

$$LR ( g ) = \frac{Pois ( k ; {s}^{avg} \cdot {λ}^{P , D} )}{Pois ( k ; {s}^{avg} \cdot {λ}^{P , {B}_{g}} )} \qquad ( 11 )$$

again with λ^{P,D}=1 for an autosomal dominant disease and λ^{P,Bg} being the expected population count of bin P variants for gene g. For example, if three bin P variants are observed with an average pathogenicity score of 0.93 in a gene g with λ^{P,Bg}=2.7, then LR(g)≈0.25. A procedure for evaluating autosomal recessive diseases in accordance with some embodiments is analogous, except that λ^{P,D}=2.

Noting that in males, hemizygous variants on the X chromosome are called as homozygous by current variant-calling software, λ^{P,D} may be set to 2 for both recessive and dominant X-chromosomal diseases.

### Identification of a Known Pathogenic Variant

There exist multiple databases of pathogenic variants in genetic disease, including ClinVar and the Human Gene Mutation Database (HGMD), which contain over one hundred thousand previously characterized pathogenic variants. If one of these variants is found, even in a gene such as TTN that is characterized by a high frequency of predicted pathogenic variants in the population, the result may be taken as being supportive of a diagnosis associated with variants in the gene. An arbitrary likelihood ratio of 1000 to 1 may be assigned in such cases.

Score for Genes with no Bin P Variants

Some embodiments of the technology described herein are designed to work whether or not genetic evidence is available to support a candidate diagnosis. If for instance, the individual being sequenced is affected by a Mendelian disease for which the causative genes have not yet been identified, then if there is a good phenotypic match, the analysis procedure described herein may include the disease in the overall results. Therefore, the genotype score may be omitted from the overall likelihood ratio score for Mendelian diseases in the HPO database that have a currently unclarified molecular basis. If the molecular basis of a disease is known to be mutations in a gene g, but no bin P variants or no variants at all are found in that gene, then a likelihood ratio score of 1/20 may be assigned for autosomal dominant diseases, reflecting an estimation that the probability of missing a pathogenic variant if one is present is about 5%. The intuition for this step is that some downweighting should be performed if no candidate variant is found in a gene but given the presumed high prevalence of false-negative results in exome/genome sequencing, it would not be desirable to radically downweight otherwise strong candidates.

### Combined Genotype-Phenotype Likelihood Ratio Score

Some embodiments of the technology described herein take as input a Variant Call Format (VCF) file and a list of HPO terms representing the set of phenotypic abnormalities observed in the individual being sequenced. For each of the ˜4,000 Mendelian diseases in the HPO database for which a causative disease gene has been identified, all predicted pathogenic (bin P) variants are extracted and their average pathogenicity score is calculated. The genotype score is then calculated based on the genotypes and predicted pathogenicities of the variant as described above. The likelihood ratios are calculated for each phenotypic feature as described above. The final likelihood ratio score for some disease Dⱼ is then:

$$LR ( {D}_{j} ) = LR ( gt ( g ) ) \times {Π}_{i} \frac{P ( {h}_{i} \vert {D}_{j} )}{P ( {h}_{i} \vert ⫬ {D}_{j} )} \qquad ( 14 )$$

### Ranking Candidates

Some embodiments of the technology described herein calculate the likelihood ratio score of equation (14) for each disease represented in the HPO disease database. The diseases are then ranked according to the posttest probability.

### Example Applications

As noted above, some embodiments take as input a VCF file from an exome, genome, or gene panel experiment in addition to a list of HPO terms (or terms from other suitable ontologies) that describe the phenotypic abnormalities of the person being investigated. The output of the processing using the techniques described herein is a ranked list of candidate diagnoses, each of which is assigned a posttest probability. Each of the phenotype ontology terms is conceived of as a diagnostic test, and a likelihood ratio is calculated for each term representing the probability that a proband has the term in question if the proband has the candidate diagnosis divided by the probability of the proband having the term if the proband does not have the candidate diagnosis. In contrast to some conventional approaches to genomic diagnosis, the technique described herein includes diseases with no known associated disease gene in the differential. However, if a disease gene is known, then a likelihood ratio is calculated for the observed genotype of the gene based on an expectation of observing one or two causative alleles according to the mode of inheritance of the disease and also the probability of observing called pathogenic variants in the gene in the general population. The individual likelihood ratios are multiplied to obtain a composite likelihood ratio, which, together with the pretest probability of each disease, is used to calculate the posttest probability which is used to rank the diseases.

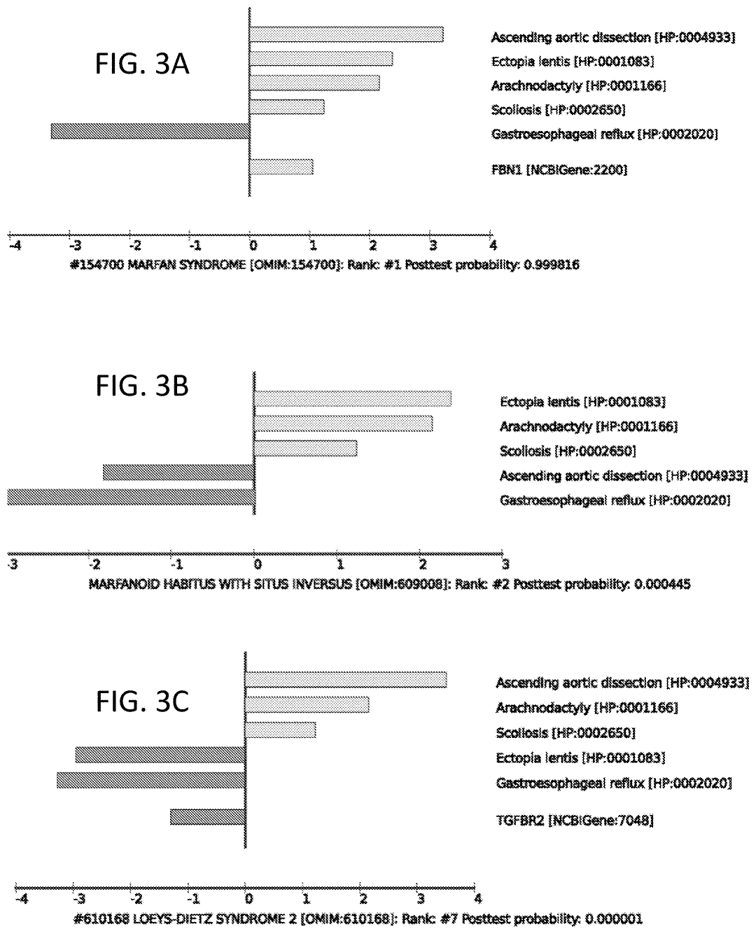

FIGS. 3A-C illustrate an application of the techniques described herein for a proband with characteristic features of Marfan syndrome (MFS), Ascending aortic aneurysm, Ectopia lentis, Arachnodactly, and Scoliosis. The feature Gastroesophageal reflux was included as a common, but unrelated (coincidental) finding to test the ability of the likelihood ratio technique to identify unrelated phenotypic findings. The results of the analysis are displayed by showing bars whose magnitude is proportional to the decadic logarithm of the likelihood ratios of each tested feature. Features that support the differential diagnosis are directed to the right of a vertical line in the center of the plot, and features that speak against the differential diagnosis are directed to the left of the center vertical line.

Given the set of input features, the likelihood ratio technique correctly identified MFS as the highest ranking candidate disease (having a posttest probability of 0.9999) from among 7000 candidate diseases. Exome sequencing in this example case revealed a heterozygous variant has been identified in the causative gene for MFS, FBN1. The graphical display of the results shown in FIG. 3A indicates how much each feature contributed to the overall prediction. Ascending aortic dissection is a relatively rare feature (with high specificity), with an LR of 1529:1. On the other hand, Scoliosis is more common and thus less specific, and has an LR of only 17.2. The LR for the coincidental finding Gastroesophageal reflux is 5.38×10⁻⁴, or roughly 1860:1 against the diagnosis as shown in FIG. 3A.

The second ranked candidate disease, Marfanoid habitus with abnormal situs, is not characterized by Ascending aortic dissection, and so the LR for this relatively specific query term substantially reduces the posttest probability of this diagnosis as shown in FIG. 3B. Marfanoid habitus with abnormal situs is an ultrarare disorder with no known disease gene, and so the genotype does not contribute to its score. In contrast, if no predicted pathogenic variant is identified in the gene associated with a candidate disease, then the genotype score may be calculated based on an estimated probability of a false-negative genotype result of 5%. This is the case for Loeys-Dietz syndrome type 2 (as shown in FIG. 3C), which is an important differential diagnosis of Marfan syndrome, but in this example receives a lower score because no mutation was identified in its associated disease gene TGFBR2.

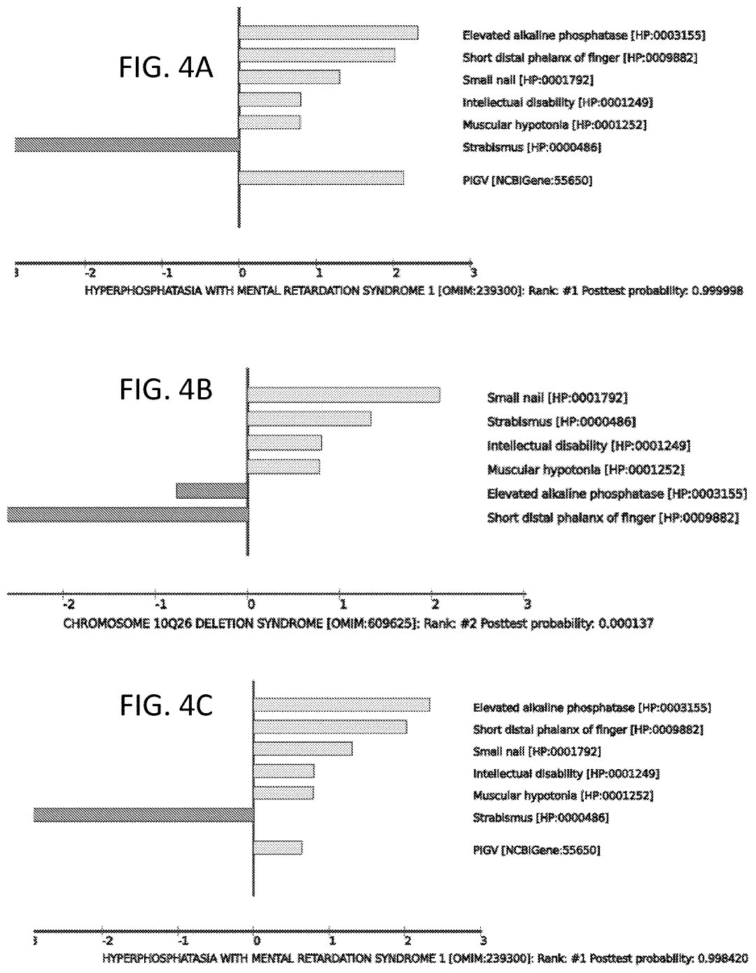

The approach for autosomal recessive diseases is analogous except that the genotype score is calculated with the expectation that two pathogenic alleles are present in affected individuals. FIG. 4A shows the results of a query with phenotypic features that are classic manifestations of hyperphosphatasia mental retardation syndrome type 1. The genotype of the biallelic predicted pathogenic variants in the corresponding disease gene PIGV leads to a higher LR score for the genotype than with a dominant disease because it is less likely to observe two predicted pathogenic variants unrelated to disease than to observe one. Strabismus (crossed eyes) was included as an unrelated term in this query.

The second best candidate, chromosome 10q26 deletion syndrome (shown in FIG. 4B), is characterized by strabismus, and accordingly FIG. 4B shows that this term is contributory in this case, but two other features are not matches for chromosome 10q26 deletion syndrome. FIG. 4C shows a simulated case in which only one predicted pathogenic variant in the disease gene for hyperphosphatasia mental retardation syndrome type 1 (PIGV) is found. Cases like this are not uncommon, and clinical judgement is required to assess whether additional investigations should be performed to identify a presumed second mutation (for instance, a structural variant that was missed by WES/WGS diagnostics). The techniques described herein assign a positive, but smaller likelihood ratio to this finding, which may be more useful than ruling out the gene because a heterozygous genotype is not causative in autosomal recessive disease.

Another benefit of the likelihood ratio approach described herein compared to conventional techniques is that the LR approach provides some information about the strength of the prediction. Given the overall diagnostic yield of exome/genome sequencing is less than 50% (depending on the study), it is expected that even the highest ranked candidate may not be a good candidate in many cases. The likelihood ratio determined in accordance with the techniques described herein provides an estimation of the strength of the prediction by means of the posttest probability, which was calculated as nearly 100% in the first two examples.

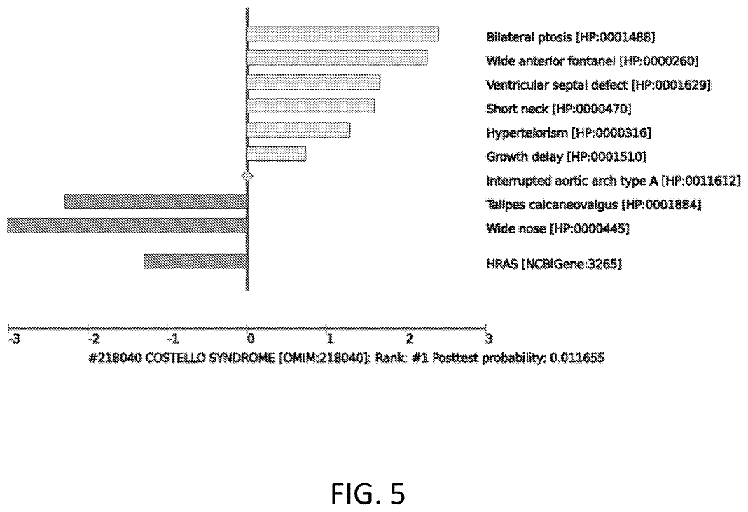

FIG. 5 shows the results of a simulated query in which no diagnosis could be established using conventional techniques. FIG. 5 shows the highest-ranked candidate disease, Costello Syndrome. Even for this top-ranked candidate, several features do not “match” the candidate diagnosis (e.g., Tallpes calcaneovalgus, Wide nose), and so the top candidate has a posttest probability of only about 1.2%. This suggests that Costello syndrome may not be the correct diagnosis and that the clinician may need to look elsewhere to continue the differential diagnostic process.

Some conventional approaches based on semantic similarity algorithms search for the best match between each query term and the terms that are used to annotate each disease in the database, and average the semantic similarity scores of each term. In contrast, the likelihood ratio score determined in accordance with the techniques described herein involves the product of an arbitrary number of individual likelihood ratios, and so in principle, adding more terms as input to the algorithm can continue to improve the composite likelihood ratio if the additional terms are good matches for the correct candidate. On the other hand, unrelated terms could reduce the likelihood ratio, and so an increased amount of noise could adversely affect the rankings.

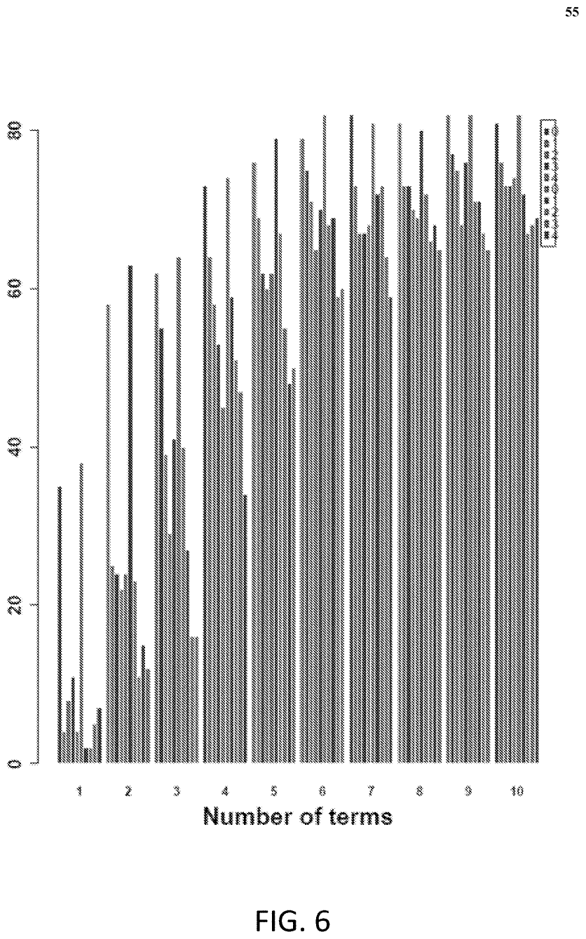

In order to test these influences, a computational simulation was performed with varying parameter settings. For each simulation, a computational proband was simulated to have a disease d with a total of N=1, . . . , 10 HPO terms that were drawn from the annotations for disease d for and from K=0, . . . , 4 unrelated (“noise”) HPO terms drawn at random from the entire ontology. If less than N terms were available for a disease d, then all of the terms annotating d were chosen. In order to simulate the effect of inexact or imprecise phenotyping, simulations in which the original terms were replaced by a parent (more general) term (the noise terms were not changed) were performed. As observed in FIG. 6, the overall performance increased with an increasing number of N terms until N=7, where even with four additional noise terms and imprecision caused by replacing original terms by their parents, the correct diagnosis was placed in first place over 50% of the time.

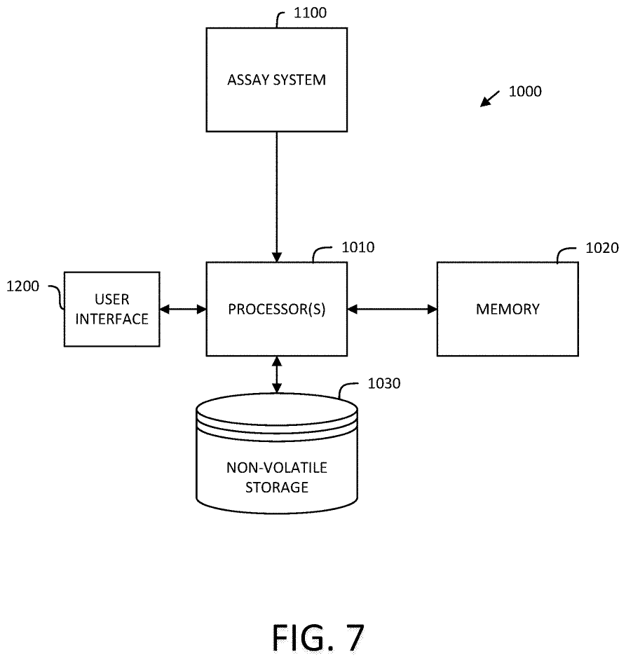

An illustrative implementation of a computer system 1000 that may be used in connection with any of the embodiments of the disclosure provided herein is shown in FIG. 7. The computer system 1000 includes one or more computer hardware processors 1010 and one or more articles of manufacture that comprise non-transitory computer-readable storage media (e.g., memory 1020 and one or more non-volatile storage devices 1030). The processor(s) 1010 may control writing data to and reading data from the memory 1020 and the non-volatile storage device(s) 1030 in any suitable manner. To perform any of the functionality described herein, the processor(s) 1010 may execute one or more processor-executable instructions stored in one or more non-transitory computer-readable storage media (e.g., the memory 1020), which may serve as non-transitory computer-readable storage media storing processor-executable instructions for execution by the processor(s) 1010.

In some embodiments, computer system 1000 also includes an assay system 1100 that provides information to processor(s) 1010. Assay system 1100 may be communicatively coupled to processor(s) 1010 using one or more wired or wireless communication networks. In some embodiments, processor(s) 1010 may be integrated with assay system in an integrated device. For example, processor(s) 1010 may be implemented on a chip arranged within a device that also includes assay system 1100.

Assay system 1100 may be configured to perform an assay on a biological sample from a patient to determine genetic information for the patient. The genetic information determined from the assay system 1100 may then be provided to the processor(s) 1010 for inclusion in a likelihood ratio clinical genomics analysis, as described above.

In some embodiments, computer system 1000 also includes a user interface 1200 in communication with processor(s) 1010. The user interface 1200 may be configured to provide a treatment recommendation to a healthcare professional based, at least in part, on the results of a likelihood ratio clinical genomics analysis output from processor(s) 1010.

The terms “program” or “software” are used herein in a generic sense to refer to any type of computer code or set of processor-executable instructions that can be employed to program a computer or other processor (physical or virtual) to implement various aspects of embodiments as discussed above. Additionally, according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, for example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term). The phraseology and terminology used herein is for the purpose of description and should not be regarded as limiting. The use of “including,” “comprising,” “having,” and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

1. A clinical decision support system, comprising:
at least one computer processor; and
at least one storage device having stored thereon, a plurality of computer-readable instructions that, when executed by the at least one computer processor performs a method comprising:
receiving phenotype information for a patient;
determining a likelihood ratio for each of the phenotype features included in the received phenotype information with respect to each of a plurality of diseases;
determining, based on the likelihood ratio for each of the phenotype features, a composite likelihood ratio for each of the plurality of diseases;
ranking the plurality of diseases based, at least in part, on the determined composite likelihood ratios; and
displaying at least some of the ranked plurality of diseases.

2. The clinical decision support system of claim 1, wherein the method further comprises:
determining, based on the determined composite likelihood ratios, a posttest probability that the patient has each of the plurality of diseases, and
wherein ranking the plurality of diseases based, at least in part, on the determined composite likelihood ratios comprises ranking the plurality of diseases based, at least in part, on the determined posttest probabilities.

3. The clinical decision support system of claim 2, wherein the method further comprises:
displaying information describing a contribution of one or more of the phenotype features to the determined posttest probability for each of the displayed plurality of diseases.

4. The clinical decision support system of claim 1, wherein the method further comprises:
determining treatment recommendation information based, at least in part, on the highest ranked disease of the plurality of ranked diseases; and
providing the determined treatment recommendation information to a user.

5. The clinical decision support system of claim 2, wherein the method further comprises:
receiving genotype information for the patient; and
determining the posttest probability based on the received genotype information.

6. The clinical decision support system of claim 5, wherein the method further comprises:
displaying information describing a contribution of the genotype information to the determined posttest probability for each of the displayed plurality of diseases.

7. The clinical decision support system of claim 5, wherein the genotype information comprises gene sequence information for the patient.

8. The clinical decision support system of claim 7, wherein the method further comprises;
estimating a pathogenicity of a gene variant included in the gene sequence, wherein estimating the pathogenicity of the gene variant is based on a computational pathogenicity score for the gene variant.

9. The clinical decision support system of claim 2, wherein method further comprises:
determining a likelihood ratio for a genotype included in the received genotype information with respect to each of the plurality of diseases, and
wherein determining the posttest probability based on the received genotype information comprises determining the posttest probability based on the determined likelihood ratio for the genotype.

10. The clinical decision support system of claim 9, wherein the method further comprises:
determining a combined genotype-phenotype likelihood ratio score based on the determined likelihood ratio for the genotype and the determined likelihood ratio for the phenotype features, and
wherein a posttest probability that the patient has each of the plurality of diseases comprises determining the posttest probability based on the combined genotype-phenotype likelihood score.

11. A method of providing clinical decision support, the method comprising:
receiving phenotype information for a patient;
determining a likelihood ratio for each of the phenotype features included in the received phenotype information with respect to each of a plurality of diseases;
determining, based on the likelihood ratio for each of the phenotype features, a composite likelihood ratio for each of the plurality of diseases;
ranking the plurality of diseases based, at least in part, on the determined composite likelihood ratios; and
displaying at least some of the ranked plurality of diseases.

12. The method of claim 11, further comprising:
determining, based on the determined composite likelihood ratios, a posttest probability that the patient has each of the plurality of diseases, and
wherein ranking the plurality of diseases based, at least in part, on the determined composite likelihood ratios comprises ranking the plurality of diseases based, at least in part, on the determined posttest probabilities.

13. The method of claim 12, further comprising:
displaying information describing a contribution of one or more of the phenotype features to the determined posttest probability for each of the displayed plurality of diseases.

14. The method of claim 11, further comprising:
determining treatment recommendation information based, at least in part, on the highest ranked disease of the plurality of ranked diseases; and
providing the determined treatment recommendation information to a user.

15. The method of claim 12, further comprising:
receiving genotype information for the patient; and
determining the posttest probability based on the received genotype information.

16. The method of claim 15, further comprising:
displaying information describing a contribution of the genotype information to the determined posttest probability for each of the displayed plurality of diseases.

17. The method of claim 15, wherein the genotype information comprises gene sequence information for the patient.

18. The method of claim 16, further comprising;
estimating a pathogenicity of a gene variant included in the gene sequence, wherein estimating the pathogenicity of the gene variant is based on a computational pathogenicity score for the gene variant.

19. The method of claim 12, further comprising:
determining a likelihood ratio for a genotype included in the received genotype information with respect to each of the plurality of diseases, and
wherein determining the posttest probability based on the received genotype information comprises determining the posttest probability based on the determined likelihood ratio for the genotype.

20. The method of claim 19, further comprising:
determining a combined genotype-phenotype likelihood ratio score based on the determined likelihood ratio for the genotype and the determined likelihood ratio for the phenotype features, and
wherein a posttest probability that the patient has each of the plurality of diseases comprises determining the posttest probability based on the combined genotype-phenotype likelihood score.

21. A non-transitory computer readable medium encoded with a plurality of instructions that, when executed by at least one computer processor perform a method, the method comprising:
receiving phenotype information for a patient;
determining a likelihood ratio for each of the phenotype features included in the received phenotype information with respect to each of a plurality of diseases;
determining, based on the likelihood ratio for each of the phenotype features, a composite likelihood ratio for each of the plurality of diseases;
ranking the plurality of diseases based, at least in part, on the determined composite likelihood ratios; and
displaying at least some of the ranked plurality of diseases.

22. The non-transitory computer readable medium of claim 21, wherein the method further comprises:
determining, based on the determined composite likelihood ratios, a posttest probability that the patient has each of the plurality of diseases, and
wherein ranking the plurality of diseases based, at least in part, on the determined composite likelihood ratios comprises ranking the plurality of diseases based, at least in part, on the determined posttest probabilities.

23. The non-transitory computer readable medium of claim 22, wherein the method further comprises:
receiving genotype information for the patient; and
determining the posttest probability based on the received genotype information.

24. The non-transitory computer readable medium of claim 23, wherein the method further comprises:
displaying information describing a contribution of the genotype information to the determined posttest probability for each of the displayed plurality of diseases.
